(19) United States
(12) Patent Application Publication
(10) Pub. No.: US 2007/0099227 A1
(43) Pub. Date: May 3, 2007

(54) Significance analysis using data smoothing with shaped response functions

(76) Inventors: Bo U. Curry, Redwood City, CA (US); Jayati Ghosh, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/607,786

(22) Filed: Nov. 30, 2006

Related U.S. Application Data
(63) Continuation-in-part of application No. 10/963,883, filed on Oct. 12, 2004.
(60) Provisional application No. 60/741,127, filed on Nov. 30, 2005.

(51) Int. Cl. C12Q 1/68; G06F 19/00

(52) U.S. Cl. 435/6; 702/20

(57) ABSTRACT

Methods, systems and computer readable media for analyzing data. A variable width window is applied to data points from the data which are ordered to correspond to locations along a chromosome of targeted nucleotide sequences whose relative abundances are represented by the data points. The window applied is a moving window, the width of which is variable upon movement of the window to capture a fixed number of the data points, wherein the width is symmetrical about the data point to which a weighted average is to be assigned. A response function is applied to the data points captured in the window. The response function is symmetrical about the data point to which a weighted average is to be assigned.

### CROSS-REFERENCE

This application is a continuation-in-part application of co-pending application Ser. No. 10/963,883, filed Dec. 14, 2004, which is incorporated herein by reference in its entirety and to which application we claim priority under 35 USC §120.

This application claims the benefit of U.S. Provisional Application No. 60/741,127, filed Nov. 30, 2005, which application is incorporated herein by reference.

### BACKGROUND OF THE INVENTION

Many events during the cell cycle can cause genomic instability through the deletion, duplication or translocation of DNA regions and result in alterations in DNA copy sequence number. Cancer progression often involves such changes in DNA copy number via over-expression of oncogenes and inactivation of tumor suppressor genes. Comparative genomic hybridization (CGH) is an important experimental technique that allows genome-wide analysis of DNA sequence copy number. The use of arrays for comparative genomic hybridization (aCGH) allows simultaneous evaluation of copy numbers at multiple positions across an entire genome, and provides a tool for clinical evaluation of cancer progression.

In a typical array CGH experiment, DNA from test cells is compared directly to DNA from normal cells. A glass slide or other array substrate is spotted with small DNA fragments from mapped genomic targets (i.e., DNA fragments of known identity and genomic position). A DNA test sample of interest and a DNA reference sample are each differentially labeled, and the combined test and reference samples are applied to the microarray. Intensity measurements for the genomic target sequences are then made to determine variations in copy number. Since the reference sample is generally diploid across the genome, target sequences with test intensities greater than the reference intensities indicate a gain in copy number, while lower intensities in the test sample indicate a loss in copy number.

Hybridization of complex genomic samples to microarrays often results in a signal-to-noise ratio that is poor for individual probes. Such noise may include compression of ratios due to aneuploidy, the existence of polymorphic sites, experimental noise, or other sources. Noise levels can make it difficult to exactly determine change points (locations where a change in copy number occurs) and the actual values of copy numbers. Thus, in analyzing array CGH data, it has become common to employ some form of data smoothing to reduce noise.

Since array CGH data is usually represented as a function of the positions of the probes along a chromosome, moving average data smoothing techniques are commonly employed. In the moving average data smoothing method, the data at each point is replaced by the average value of the point of interest and a selected number of neighboring points. Moving average data smoothing has proved to be non-optimal, however, as it tends to minimize localized copy number changes and obscure copy number changes associated with a single point on a chromosome.

There is accordingly a need for a data smoothing method that improves resolution of array CGH data, and does not minimize or obscure localized or single point copy number changes. The present invention satisfies these needs as well as others, and overcomes the deficiencies found in the background art. Similar needs exist with regard to analysis of other data types, including Location Analysis data sets, Methylation data sets or any microarray application where data from the microarray probes are ordered (e.g., sorted) by the chromosomal locations that they correspond to before analyzing the data.

Relevant Literature

Relevant literature includes U.S. Pat. No. 6,465,182; U.S. Pat. No. 6,335,167; U.S. Pat. No. 6,251,601; U.S. Pat. No. 6,210,878; U.S. Pat. No. 6,197,501; U.S. Pat. No. 6,159,685; U.S. Pat. No. 5,965,362; U.S. Pat. No. 5,830,645; U.S. Pat. No. 5,665,549; U.S. Pat. No. 5,447,841; U.S. Pat. No. 5,348,855; US2002/0006622; WO 99/23256; Pollack et al., Proc. Natl. Acad. Sci. (2002) 99: 12963-12968; Wilhelm et al., Cancer Res. (2002) 62: 957-960; Pinkel et al., Nat. Genet. (1998) 20: 207-211; Cai et al., Nat. Biotech. (2002) 20: 393-396; Snijders et al., Nat. Genet. (2001) 29:263-264; Hodgson et al., Nat. Genet. (2001) 29:459-464; Trask, Nat. Rev. Genet. (2002) 3: 769-778; Rabinovitch et al., Cancer Res. (1999) 59:5148-5153; Lee et al., Human Genet. (1997) 100:291:304; and Jong et al., Bioinformatics Advanced Access, Oxford University Press, Jul. 16, 2004. All of the above relevant literature is hereby incorporated herein, in its entirety, by reference thereto.

### SUMMARY OF THE INVENTION

Methods, systems and computer readable media are provided for smoothing data and outputting the smoothed data for use by a user. A variable width window is applied to data points from the data which are ordered to correspond to locations along a chromosome of targeted nucleotide sequences whose relative abundances are represented by the data points. The window is a moving window, and the width of the window is variable upon movement of the window to capture a fixed number of the data points with each movement of the window. The width of the window is symmetrically distributed about the data point to which a weighted average is to be assigned. A response function is applied to the data points captured in the window. The response function is symmetrical about the data point to which the weighted average is to be assigned.

The response function may have a central maximum.

The response function may be a rectangular response function.

The data provided for smoothing according to the present techniques may be obtained from a comparative genomic hybridization array.

The data provided for smoothing according to the present techniques may be location analysis data.

The data provided for smoothing according to the present techniques may be methylation data.

The response function may taper to zero on each side of a central maximum of the function.

The response function may be symmetrical in shape about the central maximum of the function.

The response function may be a Gaussian-shaped response function of the formula:

$$w ( x ) = \frac{{ⅇ}^{- {x}^{2} / ( 2 {σ}^{2} )}}{σ \sqrt{2 π}}$$

wherein σ is the 1/e width of the Gaussian, and x is data point position.

The 1/e width of the Gaussian-shaped response function may be chosen so that σ is about 1.349 times the nominal window width.

The response function may be a Lorentzian-shaped response function of the formula:

$$w ( x ) = \frac{W}{π ( {W}^{2} + {x}^{2} )}$$

wherein W is the full width half maximum of said Lorentzian-shaped response function, and x is data point position.

“W” of the Lorentzian-shaped response function may be chosen to be four times the nominal window width.

The response function may be a triangle-shaped response function.

The response function may be a biexponential response function of the formula:
w(x)=e^{-|x|/δ}/2δ,

wherein x is data point position and δ is the decay rate of the exponential.

The value for δ of the biexponential response function may be chosen to be 2 ln 2 times the nominal window width.

Significance levels may be calculated for the smoothed data obtained from applying the response function.

A graphical representation of the smoothed data may be generated and displayed.

A data analysis system is provided, including means for applying a variable width window to data points from data which are ordered to correspond to locations along a chromosome of targeted nucleotide sequences whose relative abundances are represented by the data points; means for moving the window and varying the width of the window, upon movement of the window to capture a fixed number of the data points with each movement of the window, wherein the width is symmetrically distributed about a data point to which a weighted average is to be assigned; and means for applying a response function to the data points captured in said window, wherein the shaped response function is symmetrical about the central data point in the window.

The response function may have a central maximum.

The response function may be a rectangular response function.

Further, means for generating a graphical representation of the data having been smoothed by application of the moving window and response function to the data points may be provided.

Means for displaying the graphical representation may further be provided.

The response function applied by the means for applying may be a Gaussian-shaped response function of the formula:

$$w ( x ) = \frac{{ⅇ}^{- {x}^{2} / ( 2 {σ}^{2} )}}{σ \sqrt{2 π}}$$

wherein σ is the 1/e width of the Gaussian, and x is data point position.

The 1/e width of the Gaussian-shaped response function may be chosen so that σ is about 1.349 times the nominal window width.

The response function applied by the means for applying may be a Lorentzian-shaped response function of the formula:

$$w ( x ) = \frac{W}{π ( {W}^{2} + {x}^{2} )}$$

wherein W is the full width half maximum of the Lorentzian-shaped response function, and x is data point position.

“W” of the Lorentzian-shaped response function may be chosen to be four times the nominal window width.

The response function applied by the means for applying may be a triangle-shaped response function.

The response function applied by the means for applying may be a biexponential response function of the formula:
w(x)=e^{-|x|/δ}/2δ
wherein x is data point position and δ is the decay rate of the exponential.

The value of δ of the biexponential response function may be chosen to be 2 ln 2 times the nominal window width.

A computer readable medium carrying one or more sequences of instructions from a user of a computer system for smoothing data is provided, wherein the execution of the one or more sequences of instructions by one or more processors cause the one or more processors to perform the steps of: applying a variable width window to data points from the data which are ordered to correspond to locations along a chromosome of targeted nucleotide sequences whose relative abundances are represented by the data points, wherein the window is a moving window, the width of which is variable upon movement of the window to capture a fixed number of the data points with each movement of the window, and wherein the width is symmetrically distributed about the data point to which a weighted average is to be assigned; and applying a response function to the data points captured in said window, the response function being symmetrical around the data point to which the weighted average is to be assigned.

Further, a step of generating a graphical representation of the data having been smoothed by application of the moving window and response function to the data points may be performed.

These and other features of the invention will become apparent to those persons skilled in the art upon reading the details of the methods, systems and computer readable media as more fully described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an exemplary substrate carrying an array, such as may be used for CGH, Location Analysis, Methylation or other microarray-based experiments, for example, such as may be used to extract and analyze signals using systems and methods described herein.

FIG. 2 shows an enlarged view of a portion of FIG. 1 showing spots or features.

FIG. 3 is a graphical representation of a square-shaped response function as used in prior art moving average data smoothing.

FIG. 4 is a graphical representation of a Gaussian-shaped response function for data smoothing in accordance with the invention.

FIG. 5 is a graphical representation of a Lorentzian-shaped response function for data smoothing in accordance with the invention.

FIG. 6 is a graphical representation of a triangle-shaped response function for data smoothing in accordance with the invention.

FIG. 7 is a flow chart illustrating processing data for data smoothing.

FIGS. 8A-8C illustrate potential drawbacks to use of a fixed-width window, as well as fixed-point windows used for calculating smoothing averages, and the advantages provided by the current techniques.

FIG. 9 illustrates the application of the method of FIG. 8C with an exemplary triangular weighting function.

FIG. 10 compares results of smoothing a region of a chromosome of a melanoma sample using a method of the present invention, to a method using a fixed size window.

FIG. 11 compares results of smoothing zoom-in data on a chromosome using a method of the present invention, to results of smoothing using a previously known fixed probe count method.

FIG. 12 is a schematic illustration of a computer system that may be used in performing methods described herein.

### DETAILED DESCRIPTION OF THE INVENTION

Before the present methods for array data smoothing are described, it is to be understood that this invention is not limited to particular genes or chromosomes described, as such may, of course, vary. It is also to be understood that the terminology used herein is for the purpose of describing particular embodiments only, and is not intended to be limiting, since the scope of the present invention will be limited only by the claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the target fragment” includes reference to one or more target fragments and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in many embodiments, are under 60 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide “probe” that is immobilized on a surface of a solid substrate, where the substrate can have a variety of configurations, e.g., a sheet, bead, or other structure. In certain embodiments, the collections of oligonucleotide probe elements employed herein are present on a surface of the same planar support, e.g., in the form of an array.

A “labeled population of nucleic acids” refers to mixture of nucleic acids that are detectably labeled, e.g., fluorescently labeled, such that the presence of the nucleic acids can be detected by assessing the presence of the label. A labeled population of nucleic acids is “made from” a chromosome sample, and the chromosome sample is usually employed as template for making the population of nucleic acids.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

“CGH” or “Comparative Genomic Hybridization” refers generally to techniques for identification of chromosomal alterations (such as in cancer cells, for example). Using CGH, ratios between tumor or test sample and normal or control sample enable the detection of chromosomal amplifications and deletions of regions that may include oncogenes and tumor suppressive genes, for example.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein. In certain aspects, a CGH array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing the arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences. In certain embodiments, one can have all of the probes directed to coding sequences. In certain other aspects, individual probes comprise sequences that do not normally occur together, e.g., to detect gene rearrangements, for example.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic (e.g., non-coding) regions of a nucleotide sample of interest. In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In certain aspects, longer polynucleotides may be used as probes. In addition to the oligonucleotide probes described above, cDNAs, or inserts from phage BACs (bacterial artificial chromosomes) or plasmid clones, can be arrayed. Probes may therefore also range from about 201-5000 bases in length, from about 5001-50,000 bases in length, or from about 50,001-200,000 bases in length, depending on the platform used. If other polynucleotide features are present on a subject array, they may be interspersed with, or in a separately-hybridizable part of the array from the subject oligonucleotides.

In still other aspects, probes on the array comprise at least coding sequences.

In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

“Location Analysis” is sometimes also referred to as “ChIP-on-chip technology” and refers to techniques known in the art that use microarray probes to determine the location on the genomic DNA sequence where a regulatory protein is bound. ChIP-on-chip pairs chromatin immunoprecipitation (ChIP) with microarrays (chip) to analyze how regulatory proteins interact with the genome of living cells.

“Methylation”, “methylation analysis” or DNA methylation analysis refers to techniques known in the art that use microarray probes to analyze methylated DNA sequences. DNA methylation involves the addition of a methyl group to a DNA sequence, for example, to the number 5 carbon of the cytosine pyrimidine ring. Methylation of Cytosine-Guanine (CpG) dinucleotides in genomic DNA has been correlated to gene silencing, and is regarded as one of the most significant epigenetic events.

Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

An array is “addressable” when it has multiple regions of different moieties, i.e., features (e.g., each made up of different oligonucleotide sequences) such that a region (i.e., a “feature” or “spot” of the array) at a particular predetermined location (i.e., an “address”) on the array will detect a particular solution phase nucleic acid sequence. Array features are typically, but need not be, separated by intervening spaces.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface-bound polynucleotides, as is commonly known in the art and described below, is not a mixture of capture agents because the species of surface-bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides, polypeptides and intact chromosomes of interest are well-known in the art and include, for example, ion-exchange chromatography, affinity chromatography, sorting, and sedimentation according to density.

The terms “assessing” and “evaluating” are used interchangeably to refer to any form of measurement, and include determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

If a surface-bound polynucleotide “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

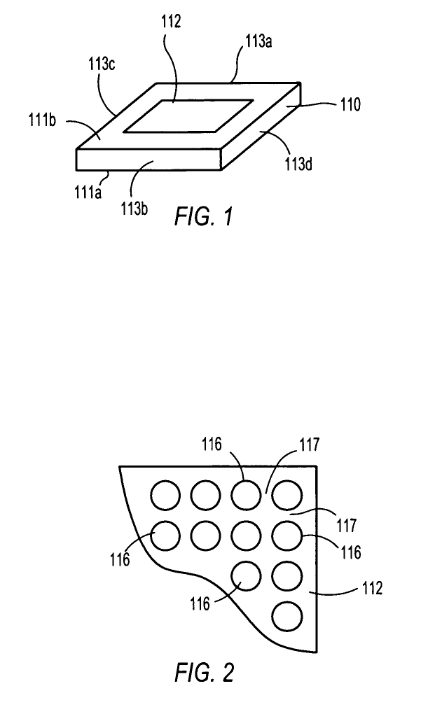

An exemplary array is shown in FIGS. 1-2, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a surface of the substrate and depending on the use of the array, any or all of the arrays may be the same or different from one another and each may contain multiple spots or features. The one or more arrays 112 usually cover only a portion of the surface 111b, with regions of the surface 111b adjacent the opposed sides 113c, 113d and leading end 113a and trailing end 113b of slide 110, not being covered by any array 112. A surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the surface 111b and the first nucleotide.

Substrate 110 may carry on surface 111a, an identification code, e.g., in the form of bar code (not shown) or the like printed on a substrate (e.g., in the form of a paper or plastic label) attached by adhesive or any convenient means. The identification code contains information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

Data Smoothing

The data smoothing methods of the invention are described in terms of use with data derived from arrays or microarrays. It should be understood, however, that the invention may be used with any data that carries genomic copy number data, including data derived from arrays, polymerase chain reaction (PCR) experiments, cell sorting, or other techniques.

The invention is particularly useful in association with arrays capable of providing genomic copy number information. Arrays suitable for use in performing the subject methods may, for example, contain a plurality (i.e., at least about 100, at least about 500, at least about 1000, at least about 2000, at least about 5000, at least about 10,000, at least about 20,000, usually up to about 100,000 or more) of addressable features containing oligonucleotides that are linked to a usually planar solid support such as a glass or silicon substrate. Features on an array usually contain polynucleotides that hybridize to, i.e., bind to, genomic sequences from a cell. Such comparative genome hybridization arrays typically include a plurality of different oligonucleotides that are addressably arrayed. The array features may also contain other polynucleotides, such as cDNAs, or inserts from phage BACs (bacterial artificial chromosomes) or plasmid clones. While the CGH arrays usually contain features of oligonucleotides, they may also contain features of polynucleotides that are about 201-5000 bases in length, about 5001-50,000 bases in length, or about 50,001-200,000 bases in length, depending on the platform used. If other polynucleotide features are present on a subject array, they may be interspersed with, or in a separately-hybridizable part of the array from, the subject oligonucleotides.

The arrays used with the invention may be prepared by a variety of well-known techniques, including drop deposition from pulse jets or from fluid-filled tips, etc, or using photolithographic means. Polynucleotide precursor units (such as nucleotide monomers), in the case of in situ fabrication, or a previously synthesized polynucleotides (e.g., oligonucleotides, amplified cDNAs or isolated BAC, bacteriophage and plasmid clones, and the like) can be deposited on arrays. Common array fabrication techniques are described in U.S. Pat. No. 6,242,266, U.S. Pat. No. 6,232,072, U.S. Pat. No. 6,180,351, U.S. Pat. No. 6,171,797, and U.S. Pat. No. 6,323,043.

The methods of the invention will be more fully understood by first considering the moving average approach to data smoothing for CGH array data. However, as already noted, the present methods are equally applicable to other types of data, including, Location Analysis data sets, Methylation data sets or any microarray application where data from the microarray probes are ordered (e.g., sorted) by the chromosomal locations that they correspond to before analyzing the data. By ordering the data of any of these types according to sequential locations on the chromosomes that they correspond to, it would be readily apparent to those of ordinary skill in the art how to apply the current methods to these other types of data, as such application would be straight forward.

When analyzing or visually representing array CGH data, it is common to represent the data as a function of the positions of the array probes (oligonucleotide features on an array surface) along each respective chromosome. In the moving average approach to data smoothing, the data at each point or probe position is replaced by the average value of the point of interest and a number of its neighboring points. The number of neighboring points depends on the type of moving average utilized. In some moving averages, the number of points that are averaged is kept fixed at, for example, 3, 5, 7, 9, 11 points, and each point is given equal weight. In other types of moving average smoothing, a window of constant width is moved across the data, and all points within the range of this window, when centered on the point of interest, are averaged to yield the moving average value for the point.

The moving average can be represented by:

$$\overline{y} = \frac{1}{( m + n + 1 )} {\sum }_{i = j - n}^{j + m} {y}_{i} \qquad ( 1 )$$

where y̅ⱼ is the moving average of the measurements yᵢ of the jth point, and j−n and j+m are the first and last points respectively in the moving average. For example, in an 11-point moving average n=m=5, and each point is the average of 11 adjacent points, centered on the sixth point. The individual measurements yᵢ may be raw signals or calculated values from microarray data, such as background-subtracted signals, dye-bias-corrected signals, normalized signals, ratios, log(ratios), log₂(ratios), or other transforms of the any of these measured values.

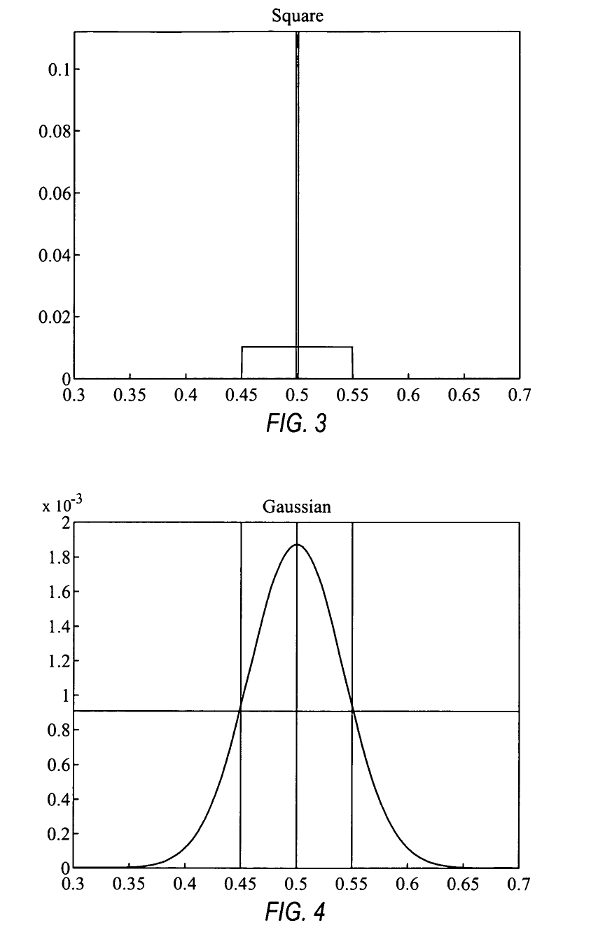

Moving average data smoothing results in a rectangular or “flat-topped” weighting or response function, as shown graphically in FIG. 3, with relative position shown on the x-axis, and relative weighting shown along the y-axis. FIG. 3 shows the moving average response function due to a single non-zero point, centered at x=0.5, generated from a data set of 1000 points on the x-axis.

The greatest drawback of moving average data smoothing is that it obfuscates the resolution of the measurement system, making the effective resolution of the average lower than the actual resolution. For example, if a one megabyte (MB) moving average is applied to an array with 100 kilobyte (kB) resolution, a lower uncertainty is gained by averaging together multiple independent measurements. This occurs, however, at the cost of losing the higher resolution of the system by averaging together points that may have independent biological variation. A single point that varies a lot from its neighbor will pull its neighbors in the direction of the change but will be held back by its neighbors as well. As a result, localized or single point changes in copy number are obscured by moving average data smoothing.

Generally, the variance of the weighted mean for a set of points can be expressed in terms of the uncertainties of each of the points as:

$$V ( {\overline{y}}_{j} ) = \frac{{\sum }_{i = j - n}^{j + m} {σ}_{i}^{2} {w}_{i}^{2}}{{( {\sum }_{i = j - n}^{j + m} {w}_{i} )}^{2}} \qquad ( 2 )$$

where σᵢ is the standard deviation of the ith point, and wᵢ is the weight assigned to the ith point.

In the case of a moving average, the weighting function has a constant value over the range of interest and is typically normalized to 1 over its range:

$${\sum }_{i = j - n}^{j + m} {w}_{i} = 1 \qquad ( 3 )$$

If the uncertainty of all data points is assumed to be equal, such as if they were all drawn from a single distribution of points, then the variance of the moving average can be expressed more simply as:

$$V ( {\overline{y}}_{j} ) = \frac{{σ}^{2}}{( m + n + 1 )} = \frac{{σ}^{2}}{{N}_{j}} \qquad ( 4 )$$

where σ is the standard deviation of the distribution of all points, and Nj is the number of points averaged for the jth point, and Nj=m+n+1.

The present invention provides data smoothing methods for CGH array data which utilize weighting or response functions that have a maximum at the center of the function, and which taper off to zero as the distance from the center of the function increases. Such weighting functions include, for example, Gaussian functions, Lorentzian functions, and triangle functions. It should be understood that any weighting function having a maximum at the center, with decreasing weighting (symmetrically or asymmetrically) at increasing distance from the center, may be used with the invention. Further, the present invention applies variable-sized windows for smoothing calculations, whose sizes depend upon the spacing between data points (e.g., probes), such that each window captures an equal number of data points.

The weighting functions used can be represented more generally by the weighting function w(x) or wᵢ. Weights are referred to as w(x) if the weight depends on the position of the ith point xᵢ by the relation:

$${\overline{y}}_{j} = \frac{{\sum }_{i = j - n}^{j + m} {y}_{i} w ( {x}_{i} )}{{\sum }_{i = j - n}^{j + m} w ( {x}_{i} )} \qquad ( 5 )$$

where each point or value yᵢ is associated with a position in space xᵢ and has an uncertainty σᵢ.

To determine the uncertainties associated with each probe on each chromosome, ideally a sufficient number of experiments would be needed to model the noise at each point, and perhaps even at different copy number values. This approach involves a fairly large number of experiments, typically more than eight (four at each value for at least two distinct known copy number values). With sufficient data, it is possible to calculate the uncertainty for each probe, as well as the intercept value (or dye-bias for two-color experiments) for each probe. In the case where an intercept is calculated on a probe-by-probe basis, yᵢ denotes the bias-corrected data. The variance for the mean for each array probe is then the square of the standard deviation for each point, σᵢ². In such a probe-specific approach the variance of the smoothed point at position xᵢ can be shown by:

$$V ( {\overline{y}}_{j} ) = \frac{{\sum }_{i = j - n}^{j + m} {σ}_{i}^{2} {w ( {x}_{i} )}^{2}}{{( {\sum }_{i = j - n}^{j + m} w ( {x}_{i} ) )}^{2}} \qquad ( 6 )$$

Unfortunately, in many cases it is impractical to carry out enough experiments to reliably measure the uncertainty of each point independently. In such cases, it is reasonable to assume that the populations from which the different array probes are drawn have equal variance, so that the variation across a set of different probes is a good estimate of the variation expected from multiple observations of a single probe. Under this assumption, all probes are assigned the same standard deviation σ, and the variance can be represented by:

$$V ( {\overline{y}}_{j} ) = \frac{{σ}^{2} {\sum }_{i = j - n}^{j + m} {w ( {x}_{i} )}^{2}}{{( {\sum }_{i = j - n}^{j + m} w ( {x}_{i} ) )}^{2}} \qquad ( 7 )$$

Referring now to FIG. 4, there is shown a graphical representation of a Gaussian-shaped weighting function usable with the methods of the invention, with relative chromosomal position shown on the x-axis, and relative weighting shown along the y-axis.

It is desirable that different choices of weighting functions result in comparable degrees of smoothing. To accomplish this, the window width is defined to be twice the horizontal distance that includes ½ of the area under the function. Unlike other measures, such as the full width at half maximum (FWHM), this measure of window width is comparable among different symmetrical weighting functions. The formula for the weights for a Gaussian function is:

$$w ( x ) = \frac{{ⅇ}^{- {x}^{2} / ( 2 {σ}^{2} )}}{σ \sqrt{2 π}} \qquad ( 8 )$$

where σ is the 1/e width of the Gaussian. The nominal window width W for this function is applied is given by:
W=2\sqrt{2}σ*erfinv(0.5)≈1.349σ (9)

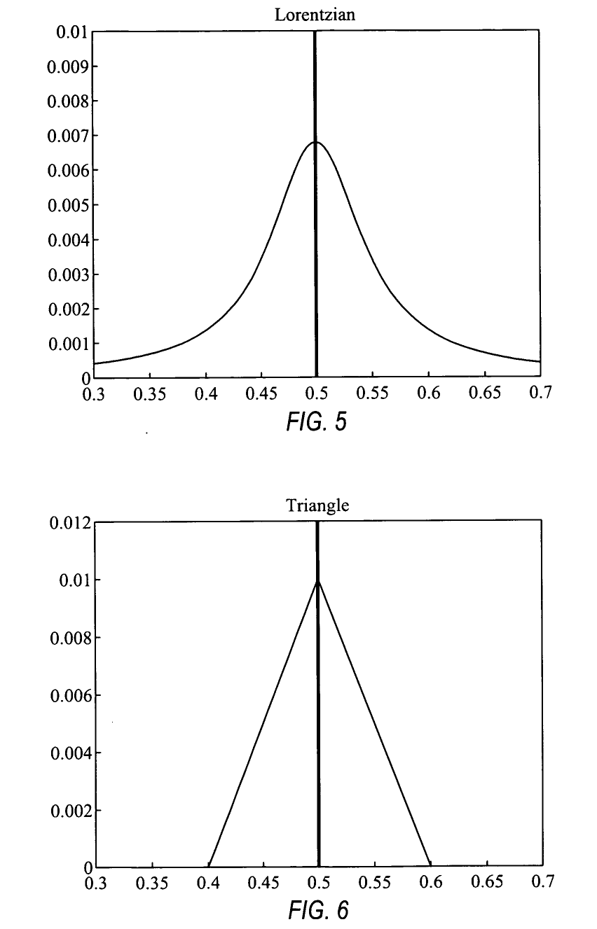

FIG. 5 graphically illustrates a Lorentzian-shaped response function usable with the methods of the invention, with relative chromosomal data point position shown on the x-axis, and relative weighting shown along the y-axis. The Lorentzian weighting function can be represented by:

$$w ( x ) = \frac{W}{π ( {W}^{2} + {x}^{2} )} \qquad ( 10 )$$

where W is the full width half maximum (FWHM) of the Lorentzian, and is four times the nominal window width.

A triangle weighting function is graphically illustrated in FIG. 6, where relative chromosomal data point position is again shown on the x-axis, and relative weighting shown along the y-axis. The triangle function has to be defined over each range:

$$w ( x ) = \{ \begin{matrix}0 , \vert x \vert > Δ \\ ( Δ - \vert x \vert ) / {Δ}^{2} , \vert x \vert \leq Δ\end{matrix} \qquad ( 11 )$$

where Δ is the x value at which the sides of the triangle intersect the x axis.

The nominal window width W of the triangle function is given by:
W=(4−2\sqrt{2})Δ (12)

Another weighting function useful with the invention, that is even more peaked than the triangle function, is the biexponential function:
w(x)=e^{-|x|/δ}/2δ (13)
The nominal window width W of the biexponential function is given by:
W=4δ ln(2) (14)

Again, it should be noted that any weighting or response functions that have a maximum at the center of the function, and which taper off as the distance from the center of the function increases, may be used with the methods of the invention, and the aforementioned weighting functions are merely exemplary of some presently preferred shaped weighting functions.

All the weighting functions are in principle applied to all data points for which the weights are nonzero. In practice, however, those weighting functions spanning an infinite domain (e.g. the Gaussian, Lorentzian, and biexponential functions) are applied over a smaller range of x values, for which the weights are significantly different from zero.

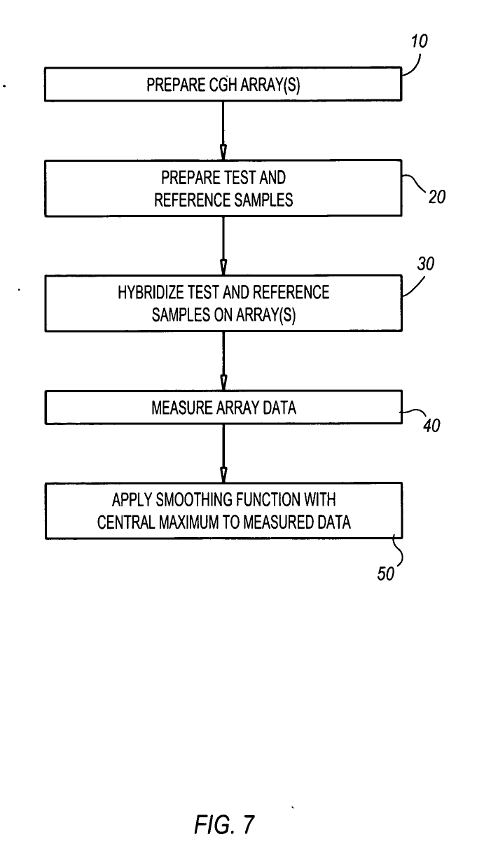

One embodiment of the subject methods is shown in the flow chart of FIG. 7. At event 10, of FIG. 7, a suitable array is prepared for generation of CGH data. Many array platforms are usable with the invention are generally well known in the art (e.g., see Pinkel et al., Nat. Genet. (1998) 20:207-211; Hodgson et al., Nat. Genet. (2001) 29:459-464; and Wilhelm et al., Cancer Res. (2002) 62: 957-960). Such arrays may contain a plurality (i.e., at least about 100, at least about 500, at least about 1000, at least about 2000, at least about 5000, at least about 10,000, at least about 20,000, usually up to about 100,000 or more) of addressable features that are linked to a usually planar solid support. Features on a subject array usually contain a polynucleotide that hybridizes with, i.e., binds to, genomic sequences from a cell. CGH arrays typically have a plurality of different BACs, cDNAs, oligonucleotide primers, or inserts from phage or plasmids, etc., that are addressably arrayed on a substrate surface. CGH arrays thus typically contain surface bound polynucleotides that are about 10-200 bases in length, about 201-5000 bases in length, about 5001-50,000 bases in length, or about 50,001-200,000 bases in length, depending on the platform used and the nature of the CGH experiment. In particular embodiments, CGH arrays containing surface-bound oligonucleotide probes, i.e., oligonucleotides of 10 to 100 nucleotides and up to 200 nucleotides in length, are particularly useful with the invention.

At event 20, test and reference samples are prepared for use with the array of event 10. This event involves obtaining and labeling test and reference genomic samples of nucleic acids. The test and reference samples may comprise, for example, the entire complement of chromosomes of a test cell and reference cell respectively (i.e., the chromosomes that make up the genome of a cell), fragmented versions thereof, amplified copies thereof, or amplified fragments thereof.

The test and reference cells used for sample preparation may be any two cells. In many embodiments, the test cell will have or be suspected of having a different phenotype compared to the reference cell. In a particular embodiment, test and reference cell pairs include cancerous cells, e.g., cells that exhibit increased genomic instability, and non-cancerous cells, respectively or cells obtained from a sample of tissue from a test subject, e.g., a subject suspected of having a chromosome copy number abnormality, and cells obtained from a normal, reference subject, respectively. Test and reference samples may be any cell of interest, including cells that contain or are suspected of containing an abnormal chromosome copy number.

The test and reference samples of nucleic acids may be labeled with the same label or different labels, depending on the actual assay protocol employed. For example, where each sample is to be contacted with different but identical arrays, the test and reference samples may be labeled with the same label. Alternatively, where both samples are simultaneously contacted with a single array, i.e., co-hybridized, to the same array, solution-phase collections or populations of nucleic acids that are to be compared are generally distinguishably or differentially labeled with respect to each other.

The test and reference nucleic acid samples may be distinguishably labeled using various well known techniques, such as primer, extension, random-priming, nick translation, and the like. See, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). “Distinguishable” labels are labels that can be independently detected and measured, even when the labels are mixed. In other words, the amounts of label present for each of the labels are separately determinable, even when the labels are co-located on the same probe feature of an array surface. Suitable distinguishable fluorescent label pairs useful with the invention include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.) and POPRO3 TOPRO3 (Molecular Probes, Eugene, Oreg.).

In certain embodiments the test and reference nucleic acid composition may be of reduced complexity (such as about 20-fold less, about 25-fold less, about 50-fold less, about 75-fold less, about 90-fold less, or at about 95-fold less complex) in terms of total numbers of sequences present in the chromosome composition as compared to the entire chromosome complements of the test and reference cells. Reduction in complexity can be achieved by using sequence specific primers in the generation of labeled nucleic acids, and by reducing the complexity of the chromosomal composition used to prepare the test and reference nucleic acid samples.

After nucleic acid purification and any pre-hybridization steps to suppress repetitive sequences (e.g., hybridization with Cot-1 DNA), the test and reference samples are hybridized onto an array or arrays in event 30. The test and reference nucleic acid samples are contacted to an array surface under conditions such that nucleic acid hybridization to the surface-bound probes can occur. The test and reference samples may be applied in a suitable buffer containing 50% formamide, 5×SSC and 1% SDS at 42° C., or in a buffer containing 5×SSC and 1% SDS at 65° C., both with a wash of 0.2×SSC and 0.1% SDS at 65° C. In many embodiments the test and reference nucleic acids may be contacted with an array surface simultaneously.

Standard hybridization techniques may be used, which may vary in stringency as desired. In certain embodiments, highly stringent hybridization conditions may be employed as described above. Kallioniemi et al., Science 258:818-821 (1992) and WO 93/18186 describe conventional CGH techniques. Several guides to general techniques are available, e.g., Tijssen, Hybridization with Nucleic Acid Probes, Parts I and II (Elsevier, Amsterdam 1993). For a description of techniques suitable for in situ hybridizations, see Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985).

Event 30 also may include post-hybridization washes to remove test and reference nucleic acids not bound to array probes in the hybridization of event 30.

In event 40, array data is measured or determined. Standard detection techniques may be used in reading the hybridization data from an array surface. Where fluorescent labeling of the test and reference nucleic acids is used, reading of the hybridized array may be achieved by illuminating the array and reading the location and intensity of resulting fluorescence at each feature of the array to detect binding complexes on the array surface. A scanner, such as the AGILENT MICROARRAY SCANNER available from Agilent Technologies, Palo Alto, Calif., may be used for measuring data. Arrays may be read by other methods such as other optical techniques (for example, detecting chemiluminescent or electroluminescent labels) or electrical techniques (where each array feature is provided with an electrode to detect hybridization). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

At event 50, a weighting or response function having a centrally located maximum is applied to the data measured in event 40. Note that, alternatively, event 50 may be carried out on array data having been received from a source, such as a data memory, for example, used to stored array data (which may be received, for example, from data storage sources including, but not limited to primary storage 204, 206, mass storage 208, CD/DVD ROM 214 or received from another data storage source via network connection 212, for example), with events 10-40 having been previously conducted, either by a user currently interested in performing the data smoothing, or by other parties. In any case, the data points are ordered to reflect the consecutive locations of the polynucleotides as they exist along a chromosome, that is, the signals from probes are arranged in an order corresponding to the consecutive order of the polynucleotides that they are designed to bind with, along a chromosome. Various weighting or response functions that have a maximum at the center of the function, and which taper off to zero as the distance from the center of the function increases, may be used with the invention as noted above. The response function is applied to data within or across a window of selected width to produce “smoothed” signals from the raw data of event 40. The smoothed data provided by use of shaped response functions with central maximum provide substantially better preservation of point-by-point variations of array data, as well as noise reduction that is necessary to see subtle effects where there are large numbers of probes, than has been achieved using previously known data smoothing methodologies. The smooth data obtained by performing event 50 may then be outputted for use by a user. Outputting may include one or more of, and is not limited to: outputting smoothed data values on a display via interface 210, outputting to a printer via interface 210 and printing smoothed data results as a hard copy output, storing smoothed data results in any of the data storage devices noted above, and transmitting smoothed data results via network connection 212.

The use of a window of fixed window with across all data points (e.g., signals from probes) in a data set for producing smoothed signals may cause a variable number of data points to be considered from window to window, such as in the case where data points are signals from probes from an aCGH array, since probes are typically not designed for polynucleotides that are located at evenly spaced locations along the chromosomes being characterized. In this case, since varying numbers of measurements contribute to each smoothed data point, the degree of statistical noise reduction also varies for each smoothed data point, which can complicate error analysis.

Another potential problem with the use of a fixed-width window for data smoothing calculations may be present when analyzing “zoom-in” arrays, where genomic regions of interest are covered much more densely than neighboring regions along the chromosome. In this case, the number of data points considered within each window, to produce each smoothed data point, can vary greatly, thus potentially varying the degree of statistical noise reduction greatly from data point to data point. For such arrays, the appropriate window size can thus vary greatly between different genomic regions, as smoothing windows which are appropriate for sparsely tiled regions will obliterate all structure in densely tiled regions, and, conversely, an appropriate window size for a densely-tiled region will perform practically no averaging in sparsely-tiled regions.

In situations such as described above, smoothing may be performed using a window that changes in width for each calculation, where necessary, to ensure that the same number (i.e., a fixed number) of data points are considered within each window, for each smoothing calculation, regardless of the total range of sequence spanned by the probes from which the data points are taken. Also, a shaped weighting function (response function) may be applied to the data points in the window to weight data points nearest the point to be smoothed more heavily than data points relatively further away from the data point to be smoothed.

Specific examples of shaped, weighted response functions include a Gaussian-shaped function as shown by equation (8), a Lorentzian-shaped function as shown by equation (10), and a triangle-shaped function defined by the ranges of equation (11). The Gaussian-shaped response function may be applied to data across a window of width W (which varies, if required, for each calculation, to ensure that a fixed number of data points are considered for each calculation) as defined by equation (9). The Lorentzian-shaped response function may be applied to data across a window of width to ensure consideration of a fixed number of data points, and wherein the response function is such that the width of the window is equal to the FWHM of the Lorentzian function. The triangle-shaped response function may be applied to data across a window of width W as defined by equation (12), wherein the width W varies, if required, for each calculation, to ensure that a fixed number of data points are considered for each calculation.

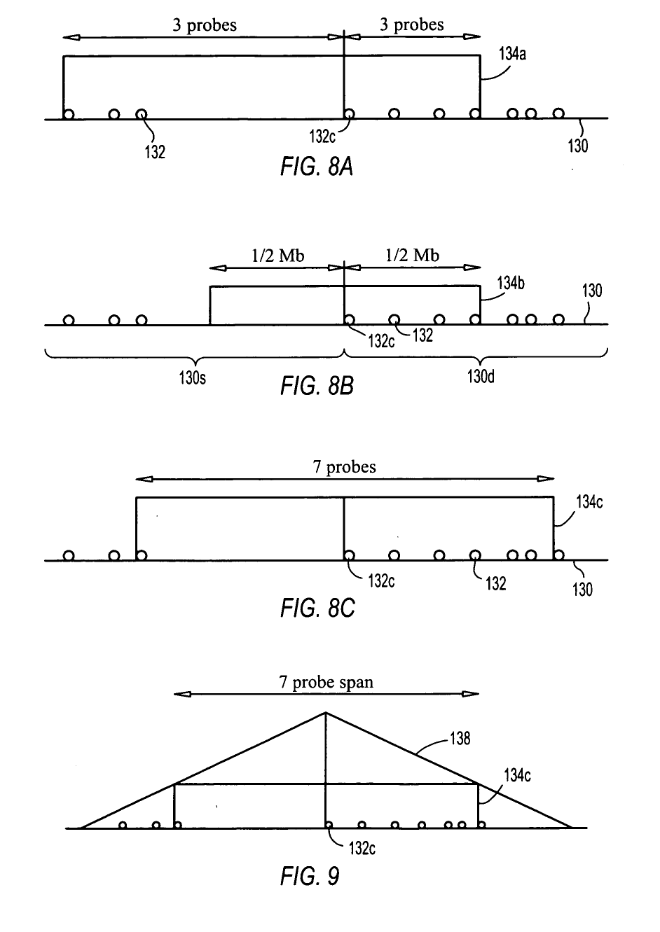

FIGS. 8A-8C illustrate potential drawbacks to use of a fixed-width window, as well as fixed-point windows used for calculating smoothing averages, and advantages provided by the current techniques. In each of FIGS. 8A-8C, line 130 schematically represents a chromosome and 132 indicates probes from which the data signals to be processed (smoothed) are read, and which are schematically shown in the locations on the chromosomes where the polynucleotides are located that hybridize to the probes 132, respectively. In each of FIGS. 8A-8C, chromosome 130 is probed by a number of probes 132 at varying intervals along the chromosome 130.

FIG. 8A illustrates use of a window to capture a fixed number of points. However, in this case, probes (data points) far from the data point for which the smoothed average is to be calculated (i.e., the central data point (probe) 132c, are weighted the same as data points/probes 132 near to point 132c. The window 134a is established by starting from a first data point/probe and extending the window until it captures the fixed number of data points. The middle data point is the data point for which the smoothed average is calculated, however, the middle data point 132c is not necessarily in the center of the window 134a, as is illustrated in the example shown.

FIG. 8B illustrates use of a window 134b of fixed width, such that each smoothing calculation performed is with regard to whatever number of probes 132 fall within the window 134b at the position that it lies when the calculation is being performed, where the window is incremented along positions on the chromosome with successive calculations, but does not change width. In the location shown in FIG. 8B it can be observed that only four probes will be considered for the smoothing calculation for data point 132c, and all probes are to the right of probe 132c. Thus, the averaging includes many more points in the dense region 130d than in the sparse region 130s. In fact, no probes from the sparse region 130s are considered for averaging the signal value of 132c in the position of window 134b shown in FIG. 8B.

In FIG. 8C, the window 134c is a fixed data point type of window, meaning that the width of the window will vary, depending upon its placement along the chromosome 130 to capture a fixed number of data points/probes. In the example shown, the fixed number is seven, and a symmetric window 134c is enlarged, starting from the central data point 132c for which the smoothed calculation is to be performed, until the fixed number of points are contained within the window 134c. Thus the window width is the smallest-width window, symmetrical about the data point 132 to be averaged, that includes the specified fixed number of points. These points may be located on one side of the point 132c to be averaged, or on both, depending upon the probe density around the point 132c to be averaged. The points contained within window 134c may be smoothed using a rectangular weighting function 134c, or may be weighted according to a shaped, tapering smoothing function, according to their distances from the point 132c.

FIG. 9 illustrates the application of the method of FIG. 8C to an exemplary triangular weighting function 138. As noted above, the window is drawn initially from the central data point (probe) 132c and is expanded symmetrically in the left and right directions to increase the width thereof until the specified number of probes (data points) are included in the window 134c. The weights assigned to the log ratio values of probes 132 are given by the equation:
w(x)=(Δ−|x|)/Δ² (15)
where the span of the triangle is determined by the effective window width, W of window 134c, of the symmetrical region spanning the specified number of points by the equation:
Δ=W/(4−2√2) (16)
Other symmetrical weighting functions can be used, as described above.

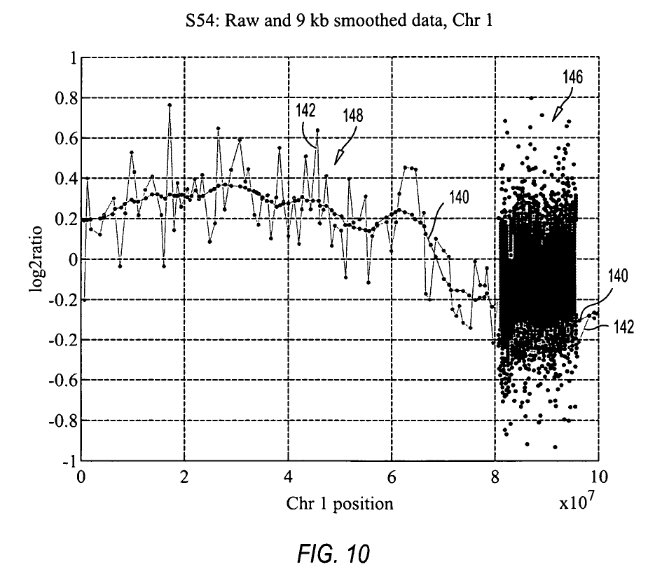

FIG. 10 compares results of smoothing a region of chromosome 1 of a melanoma sample using a current method 140 of using a variable width window to maintain a fixed number of data points in each window, as described above with regard to FIG. 8C, together with a weighting function as described herein (in this example, a triangular weighting function was used), to the results 142 of smoothing using a comparable fixed size window, wherein a triangular weighting function was also applied. The results 140 from the current method, unlike the results 142 from the fixed-window method, results in a comparable degree of smoothing regardless of the local probe density. Thus, while the fixed window method results 142 show an effective amount of smoothing in the dense region due to a sufficient number of data points captured by each fixed window in this region, in the sparse region 148, the fixed window method results 142 show very little smoothing as the fixed window is incapable of capturing a sufficient number of data points for each calculation to produce effective smoothing. In contrast, the results 142 using the present method show good smoothing in both regions 146 and 148.

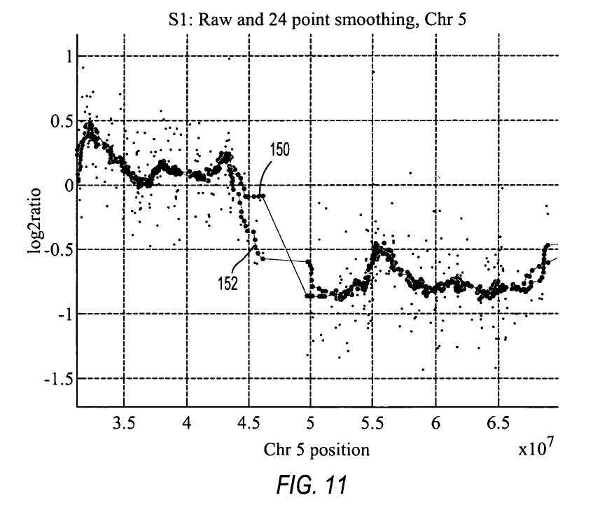

FIG. 11 compares results 150 of smoothing zoom-in data on a chromosome, using the fixed point method described with regard to FIG. 10 with a rectangular smoothing function, to the results 152 of smoothing using a previously known fixed probe count method, in which the average of each fixed number of points (in this case, the fixed number is 24) is assigned to the central point, rather than to the center of a symmetrical window. Thus the value of each point is assigned the value of the mean of the values of the twelve preceding and eleven following points. Although the methods yield comparable results 150,152 in regions where the probe density is relatively constant, the results 150 of the current method are superior in regions where the copy number changes across a gap of low probe density.

Referring again to FIG. 7, event 50 may additionally include calculation of significance level for the data smoothed using a shaped response function with a central maximum. In cases where the probes within the range of interest can be assumed to have equal variance (i.e., noise distribution is equal across the range or chromosome of interest), all probes can be assigned the same standard deviation, and the variance of each smoothed data point can be obtained using equation (7). Where equal variance for the probe range of interest cannot be assumed, and where suitable data is available, the probe-specific variance of equation (6) may be applied to the smoothed data.

Event 50 may include creating a graphical representation of the smoothed data and such graphical representation may be outputted, stored and/or transmitted in any of the manners described above.

Prior to the data smoothing of event 50, raw data from event 40 may be globally normalized such that the normalized test and reference signals are equal on average for a specified subset of “normalization” probes. Alternatively, test signals may be normalized to data obtained from controls (e.g., internal controls produce data that are predicted to be equal in value in all of the data groups). Such normalization may involve multiplying each numerical value for one data group by a value that allows the direct comparison of those amounts to amounts in a second data group. Several normalization strategies have been described (Quackenbush et al, Nat. Genet. 32 Suppl:496-501, 2002, Bilban et al Curr Issues Mol. Biol. 4:57-64, 2002, Finkelstein et al, Plant Mol Biol. 48(1-2):119-31, 2002, and Hegde et al, Biotechniques. 29:548-554, 2000). Specific examples of normalization suitable for use in the subject methods include linear normalization methods, non-linear normalization methods, e.g., using Lowess local regression to paired data as a function of signal intensity, signal-dependent non-linear normalization, qspline normalization and spatial normalization, as described in Workman et al., Genome Biol. 2002 3, 1-16. In certain embodiments, the numerical value associated with a feature signal is converted into a log number, either before or after normalization occurs.

Also prior to the data smoothing of event 50, normalized signals may be converted into log ratios of the normalized test signals to the normalized reference signals, or vice versa. In the preferred embodiment, the smoothing function of event 50 is applied to such normalized log ratios.

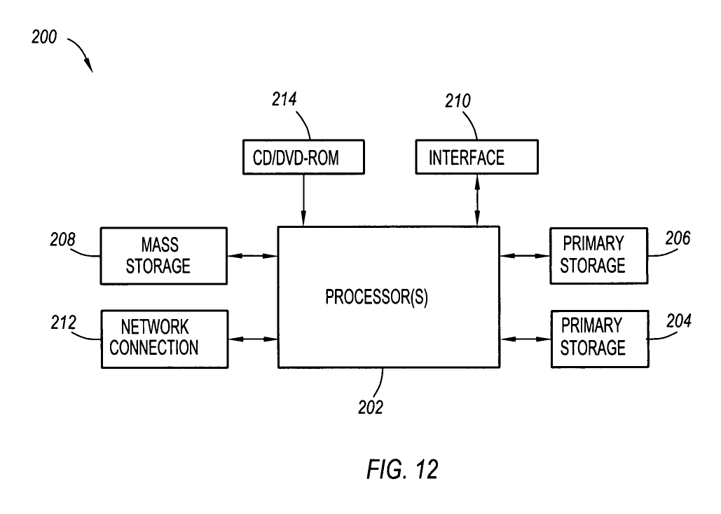

The data smoothing of event 50 will usually be embodied in logic that resides in a computer-readable medium associated with a computer system such as system 100 shown in FIG. 12. The computer system 200 includes any number of processors 202 (also referred to as central processing units, or CPUs) that are coupled to storage devices including primary storage 204 (typically a random access memory, or RAM), primary storage 206 (typically a read only memory, or ROM). As is well known in the art, primary storage 204 acts to transfer data and instructions uni-directionally to the CPU, and primary storage 206 is used typically to transfer data and instructions in a bi-directional manner Both of these primary storage devices may include any suitable computer-readable media containing program elements capable of performing the data smoothing operations described above.

A mass storage device 208 is also coupled bi-directionally to CPU 202 and provides additional data storage capacity and may include any of the computer-readable media described above. Mass storage device 208 may be used to store programs, data and the like and is typically a secondary storage medium such as a hard disk that is slower than primary storage. It will be appreciated that the information retained within the mass storage device 208, may, in appropriate cases, be incorporated in standard fashion as part of primary storage 206 as virtual memory. A specific mass storage device such as a CD-ROM 214 may also pass data uni-directionally to the CPU 202.

CPU 202 is also coupled to an interface 210 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 202 optionally may be coupled to a computer or telecommunications network using a network connection as shown generally at 212. With such a network connection, it is contemplated that the CPU 202 might receive information from the network, or might output information to the network in the course of performing the above-described method steps. The above-described devices and materials will be familiar to those of skill in the computer hardware and software arts.

The hardware elements described above may implement the instructions of multiple software modules for performing the data smoothing operations of this invention. For example, instructions for applying various shaped response functions having a central maximum to raw or normalized array data, for determining window widths to achieve fixed numbers of data points, for determining variances for smoothed data points, and for generating graphical representations of smoothed data, may be stored on mass storage device 208 or 214 and executed on CPU 202 in conjunction with primary memory 206.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as “floptical” disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter. Computer readable media is not intended to include carrier waves.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method for smoothing data, comprising:
applying a variable width window to data points from the data which are ordered to correspond to locations along a chromosome of targeted nucleotide sequences whose relative abundances are represented by the data points, wherein the window is a moving window, the width of which is variable upon movement of the window to capture a fixed number of the data points, wherein the width is symmetrically distributed about the data point to which a weighted average is to be assigned; and
applying a response function to the data points captured in said window, said response function being symmetrical about the data point to which the weighted average is to be assigned to calculate smoothed data values; and
outputting said smoothed data values.

2. The method of claim 1, wherein said response function has a central maximum.

3. The method of claim 1, wherein said response function is a rectangular response function.

4. The method of claim 1, wherein said data comprises comparative genomic hybridization data.

5. The method of claim 4, wherein said comparative genomic hybridization data is obtained from a comparative genomic hybridization array.

6. The method of claim 1, wherein said data comprises Location Analysis data.

7. The method of claim 1, wherein said data comprises Methylation data.

8. The method of claim 2, wherein said response function tapers to zero on each side of said central maximum.

9. The method of claim 2, wherein said response function is symmetrical in shape about said central maximum.

10. The method of claim 1, wherein said response function is a Gaussian-shaped response function of the formula:
$$w ( x ) = \frac{{ⅇ}^{- {x}^{2} / ( 2 {σ}^{2} )}}{σ \sqrt{2 π}}$$
wherein σ is the 1/e width of the Gaussian, and x is data point position.

11. The method of claim 10, wherein the 1/e width of said Gaussian-shaped response function is chosen so that σ is about 1.349 times the nominal window width.

12. The method of claim 1, wherein said response function is a Lorentzian-shaped response function of the formula.
$$w ( x ) = \frac{W}{π ( {W}^{2} + {x}^{2} )}$$
wherein W is the full width half maximum of said Lorentzian-shaped response function, and x is data point position.

13. The method of claim 12, wherein W of said Lorentzian-shaped response function is chosen to be four times the nominal window width.

14. The method of claim 1, wherein said response function is a triangle-shaped response function.

15. The method of claim 1, wherein said response function is a biexponential response function of the formula:
w(x)=e^{-|x|/δ}/2δ
wherein x is data point position and δ is the decay rate of the exponential.

16. The method of claim 15, wherein δ of said biexponential response function is chosen to be 2 ln 2 times the nominal window width.

17. The method of claim 1, further comprising calculating significance levels for smoothed data obtained from applying said response function.

18. The method of claim 1, further comprising generating a graphical representation of said smoothed data.

19. A comparative genomic hybridization array data analysis system, comprising:
means for applying a variable width window to data points which are ordered to correspond to locations along a chromosome of targeted nucleotide sequences whose relative abundances are represented by the data points;
means for moving the window and varying the width of the window, upon movement of the window to capture a fixed number of the data points with each movement of the window, wherein the width is symmetrically distributed about the data point to which a weighted average is to be assigned; and
means for applying a response function to the data points captured in said window, said response function being symmetrical around the central point of said fixed number of points in the window.

20. The system of claim 19, wherein said response function has a central maximum.

21. The system of claim 19, wherein said response function is a rectangular response function.

22. The system of claim 19, further comprising means for generating a graphical representation of the data having been smoothed by application of said moving window and response function to the data points.

23. The system of claim 19, wherein said response function is a Gaussian-shaped response function of the formula:
$$w ( x ) = \frac{{ⅇ}^{- {x}^{2} / ( 2 {σ}^{2} )}}{σ \sqrt{2 π}}$$
wherein σ is the 1/e width of the Gaussian, and x is data point position.

24. The system of claim 23, wherein the 1/e width of said Gaussian-shaped response function is chosen so that σ is about 1.349 times the nominal window width.

25. The system of claim 19, wherein said response function is a Lorentzian-shaped response function of the formula:
$$w ( x ) = \frac{W}{π ( {W}^{2} + {x}^{2} )}$$
wherein W is the full width half maximum of said Lorentzian-shaped response function, and x is data point position.

26. The system of claim 25, wherein W of said Lorentzian-shaped response function is chosen to be four times the nominal window width.

27. The system of claim 19, wherein said response function is a triangle-shaped response function.

28. The method of claim 19, wherein said response function is a biexponential response function of the formula:
w(x)=e^{-|x|/δ}/2δ
wherein x is data point position and δ is the decay rate of the exponential.

29. The method of claim 28, wherein δ of said biexponential response function is chosen to be 2 ln 2 times the nominal window width.

30. A computer readable medium carrying one or more sequences of instructions from a user of a computer system for smoothing data, wherein the execution of the one or more sequences of instructions by one or more processors cause the one or more processors to perform the steps of:
applying a variable width window to data points from the data which are ordered to correspond to locations along a chromosome of targeted nucleotide sequences whose relative abundances are represented by the data points, wherein the window is a moving window, the width of which is variable upon movement of the window to capture a fixed number of the data points with each movement of the window, and wherein the width is symmetrically distributed about the data point to which a weighted average is to be assigned;
applying a response function to the data points captured in said window, said response function being symmetrical around the data point to which the weighted average is to be assigned;
calculating smoothed data; and
outputting the smoothed data.

31. The computer readable medium of claim 30, wherein the following further step is performed: generating a graphical representation of the data having been smoothed by application of said moving window and shaped response function to the data points.
